(19) United States
(12) Patent Application Publication
(10) Pub. No.: US 2009/0305387 A1
(43) Pub. Date: Dec. 10, 2009

(54) COMPOSITIONS AND METHODS FOR CLEANING SURFACES

(75) Inventor: Sean Farmer, Miami Beach, FL (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Ganeden Biotech, Inc., Mayfield Heights, OH (US)

(21) Appl. No.: 12/363,442

(22) Filed: Jan. 30, 2009

Related U.S. Application Data
(60) Provisional application No. 61/062,956, filed on Jan. 30, 2008.

(51) Int. Cl. C12N 1/20

(52) U.S. Cl. 435/252.5; 435/264

(57) ABSTRACT

Cleaning compositions using the supernatant of lactic acid-producing bacteria are provided. Use of the compositions for disinfection/sanitization of surfaces are also disclosed.

### RELATED APPLICATIONS

This application claims priority to U.S. provisional application No. 61/062,956, filed Jan. 30, 2008, the contents of which is hereby incorporated by reference in its entirety.

### FIELD OF THE INVENTION

The present application relates to cleaning compositions.

### BACKGROUND OF THE INVENTION

Proper hygiene and microbial control are important in food processing plants and hospitals. Current automated processing plants place increased demands on processing equipment and cleaning procedures to ensure that microbial control is maintained throughout the process in order to prevent contamination of large amounts of processed food. Similarly, in the medical industry, bacterial control is particularly important due to the necessity to prevent cross-contamination that may lead to infection. Each time medical devices are used, they must be thoroughly cleaned by hand, or in an automatic washer, and must thereafter be dried, deodorized and sterilized. Thus, there is a pressing need for more effective cleaning solutions for environmental surfaces.

### SUMMARY OF THE INVENTION

The invention is directed to the surprising discovery that the supernatant of lactic acid-producing bacteria, particularly of Bacillus species, effectively kills pathogenic microorganisms on an environmental surface. For example, the surface is a pervious surface such as a sponge, fabric (e.g., surgical gauze), concrete or non-vitrified ceramic surface. Alternatively, the surface is an impervious surface such a metallic surface, e.g., stainless steel, titanium, or vitrified ceramic. Other surfaces include metallic surfaces such as copper, bronze, gold, silver and tin surfaces or stone surfaces such as granite, marble, soapstone, and slate surfaces. For example, the composition is used to contact medical devices, implants, or tools, and compositions from which they are manufactured, as well as food processing tools, equipment, and devices as well as the compositions from which they are manufactured. The invention describes supernatant-containing cleaning compositions. Specifically, the invention provides methods for cleaning a surface comprising contacting the surface with a Bacillus coagulans supernatant, lysate or secreted product composition. In one aspect, the Bacillus coagulans supernatant, lysate or secreted product reduces the concentration, amount, or growth of pathogenic (and/or potentially opportunistic) microorganisms on the environmental surface, thereby disinfecting the surface. By opportunistic microorganism is meant a microorganism that is usually harmless but can become pathogenic when the host's resistance to disease is impaired. Preferably, the Bacillus coagulans supernatant, lysate or secreted product is sprayed onto the surface. Alternatively, the Bacillus coagulans supernatant, lysate or secreted product is applied to the surface with a brush, cloth, sponge, or other means to disperse the supernatant, lysate or secreted product over the surface of the object to be cleaned. In another aspect, the Bacillus coagulans supernatant, lysate or secreted product is applied to the surface as a dry powder.

In one aspect, the Bacillus coagulans supernatant is used to kill on contact, i.e., the composition disinfects the surface within seconds (e.g., 1, 2, 3, 4, 5, 8, 10, 12, or 14 seconds). In another aspect, the composition remains on the surface as a liquid film and is allowed to dry for a short period of time. Alternatively, the Bacillus coagulans supernatant remains on the surface as a liquid film for a period of time before removing the supernatant from the surface. Optionally, the period of time is between about 15 seconds and about 30 minutes, e.g., between about 30 seconds and about 10 minutes; between about 1 minute and about 8 minutes; or between about 2 minutes and about 6 minutes. Preferably, the period of time is 5 minutes.

In one aspect, the invention provides agitating the Bacillus coagulans supernatant after contact with the surface. Preferably, agitation of the Bacillus coagulans supernatant after contact with the surface assists in the reduction of pathogenic microorganisms on the surface. In one aspect, the supernatant is manually agitated. Optionally, the agitation of the surface film is mediated by a device, e.g., a shaker or a sonicator.

The method is effective to disinfect surfaces that contain pathogenic microorganisms including, but not limited to: Escherichia, Enterococcus, Staphylococcus, Clostridium, Campylobacter, Pseudomonas, and Streptococcus. More specifically, exemplary pathogenic microorganisms include Bacillus cereus, Escherichia coli, Staphylococcus aureus, Vancomycin resistant Enterococcus, multi-drug resistant Enterococcus, Enterococcus faecium, Serratia marcescens, Proteus mirabilis, Klebsiella pneumoniae, and Staphylococcus epidermidis. Alternatively, the pathogenic microorganisms are fungal pathogens, such as Candida and Trichophyton. Other medically-relevant pathogens are also included by the invention.

In one aspect, the invention provides for an undiluted Bacillus coagulans supernatant. Alternatively, the Bacillus coagulans supernatant is diluted about 1:2; about 1:10; or about 1:100. Preferably, the Bacillus coagulans supernatant is diluted in deionized water. Optionally, the supernatant is in a dry powder form and reconstituted (activated) in water before use.

The invention also provides that the Bacillus coagulans supernatant is combined with one or more other surface-active disinfectants prior to contacting the surface. Suitable surface-active disinfectants include ethyl alcohol, methyl alcohol, quaternary ammonium chloride compounds, and phenolics.

The cleaning compositions of the invention are used to clean any surface. In one aspect, the surface is a surface associated with food-processing equipment, such as a meat processor, a dairy product (e.g., milk, cheese, yogurt) processing line equipment, or brewing equipment such as a continuous brewing system. Alternatively, the surface is a surface associated with medical equipment, such as fiberscopes, endoscopes, medical supplies or other materials or types of equipment that could be damaged by heat or harsh chemical disinfection. Other suitable surfaces include surfaces in restaurants, homes, nursing homes, and private and public restrooms, e.g., desks, tables, chairs, stovetops, countertops, kitchen appliances, refrigerators, shelves, floors, toilets, sinks, bathtubs, windows, dog kennels, and baby equipment, such as baby changing tables, and diaper pails. Preferably, the cleaning compositions described by the invention are used as household cleaners.

In one aspect, the cleaning composition sterilizes the surface, removing microorganisms from or killing microorganisms at or on a surface. Alternatively, the cleaning disinfects the surface, destroying microorganisms and pathogens. The invention also provides that the Bacillus coagulans supernatant has a bacteriostatic/fungistatic effect on the pathogenic microorganisms on the surface, preventing further growth of bacteria. In another aspect, the Bacillus coagulans supernatant has a bacteriocidal/fungicidal effect on the pathogenic microorganisms on the surface, destroying the bacteria. By bacteriostatic or fungistatic is meant that the proliferation of bacteria or fungi is inhibited. For example, proliferation is inhibited by 10%, 20%, 50%, 75%, or 100%. By bacteriocidal or fungicidal is meant that the bacterial or fungal cell is killed.

Preferably, the Bacillus coagulans supernatant reduces the level of pathogenic microorganisms on the surface by at least about 80%, e.g., at least about 90%, at least about 95%, or at least about 99%.

The invention provides for a composition for cleaning a surface comprising an antimicrobial amount of a supernatant of Bacillus coagulans. In another aspect, the invention provides for compositions for cleaning a surface comprising an antimicrobial amount of a secreted product of Bacillus coagulans. In yet another aspect, the invention provides for a composition for cleaning a surface comprising an antimicrobial amount of a lysate of Bacillus coagulans. The invention also provides for a composition for cleaning a surface comprising an antimicrobial amount of a peptide or organic molecule isolated from the Bacillus coagulans supernatant. Preferably, the compositions described by the invention reduce the level of pathogenic microorganisms on a surface. Optionally, the cleaning compositions contain a supernatant or composition secreted by Pseudomonas lindbergii.

The invention also provides for compositions for cleaning a surface comprising an antimicrobial amount of a supernatant of Bacillus coagulans and a surface-active agent. In one aspect, the surface-active agent is selected from the group consisting of ethyl alcohol, methyl alcohol, and a quaternary ammonium chloride compound. Optionally, the composition is in the form of a dry powder, pellets or particles. The pellets or particles are about 1 μm to about 50 mm in size, e.g., about 1 μm to about 10 μm; about 10 μm to about 100 μm; about 100 μm to about 1 mm; about 1 mm to about 10 mm; or about 10 mm to about 50 mm. Alternatively, the composition is in the form of a liquid or suspension, e.g., a solution of extracelluar products of the bacterium or a suspension of bacterial vegetative cells or spores in an aqueous medium. The particles in suspension are about 1 ηm to about 10 ηm; about 10 ηm to about 100 ηm; about 100 ηm to about 1 μm; about 1 μm to about 10 μm; about 10 82 m to about 100 μm; about 100 μm to about 1 mm.

The invention also provides for compositions for cleaning a surface comprising Bacillus coagulans spores and a surface-active agent. Optionally, the surface-active agent is trisodium phosphate (TSP), a suitable TSP substitute, bleach, alkyl dimethyl benzyl ammonium chloride, 2-butoxyethanol, sodium dichloro-s-triazinetrione dehydrate, or triclosan.

Bacillus coagulans bacteria are included in the cleaning compositions of this invention. Bacterial species include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20, ATCC Designation Number PTA-6085; GBI-30, ATCC Designation Number PTA-6086; and GBI-40, ATCC Designation Number PTA-6087; see U.S. Pat. No. 6,849,256 to Farmer).

Also within the invention is a purified compound, e.g., a protein or small organic molecule (e.g., less than 1,000 daltons molecular mass), which is characterized as having at least 50%, 75%, 95%, 100%, 2-fold, 5-fold, 10-fold or more antimicrobial activity compared to B. coagulans supernatant. The compound is identified as being a component of a B. coagulans supernatant. The compound is purified or isolated using chemical, biochemical, or recombinant techniques. The compositions described herein are substantially pure. By “substantially pure” is meant a protein, polypeptide, or other molecule that has been separated from the components that naturally accompany it. Typically, the polypeptide is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

The Bacillus coagulans bacterium or spore is purified or substantially purified. By “purified” or “substantially purified” is meant a Bacillus coagulans bacterium or spore that is substantially free of other bacterial strains, extraneous cellular material or other contaminating macromolecules, e.g., polysaccharides, nucleic acids, or proteins, from the source from which the Bacillus coagulans bacterium or spore is derived. For example, a pure or substantially pure Bacillus coagulans bacterium or spore may be obtained by extraction from a natural source. Typically, the Bacillus coagulans bacterium or spore is pure or substantially pure when it is at least 60%, 70%, 80%, 90%, 95%, or even 99% of the specified bacterial strain, by weight, free from other bacterial strains, extraneous cellular material or other contaminating macromolecules. The Bacillus coagulans bacterium or spore is purified or isolated using methods known to those skilled in the art.

Cited publications are incorporated herein by reference. Both the foregoing general description and the following detailed description and examples are exemplary and explanatory only and are not restrictive of the invention as claimed.

### BRIEF DESCRIPTION OF THE DRAWINGS

FIG. 1 is a chart demonstrating the ability of Bacillus coagulans supernatant to kill various pathogenic microorganisms.

FIG. 2 illustrates, in tabular form, a comparison of the anti-mycotic, Fluconazole with Bacillus coagulans and Pseudomonas lindbergii supernatants (generically designated Ganeden Supernatant) in the inhibition of various bacterial, fungal, and yeast species.

### DETAILED DESCRIPTION OF THE INVENTION

The present invention is directed to the discovery that the supernatant of non-pathogenic lactic acid-producing bacteria (i.e., “lactic acid bacteria”), such as the exemplary Bacillus coagulans, reduces pathogenic microorganisms on a surface.

### Probiotic Lactic Acid-Producing Bacteria

A probiotic lactic acid-producing bacteria suitable for use in the methods and compositions of the invention as defined for use in the present invention produces acid and is non-pathogenic. There are many suitable bacteria identified as described herein, although the invention is not limited to currently known bacterial species insofar as the purposes and objectives of the bacteria is described. The property of acid production is important to the effectiveness of the probiotic lactic acid-producing bacteria of this invention.

The invention provides using a lactic acid-producing bacteria, such as a spore-forming Bacillus species, such as B. coagulans. Preferably, the spore-forming Bacillus species of the invention is B. coagulans Hammer.

Exemplary methods and compositions are described herein using Bacillus coagulans. The supernatant of purified and/or isolated Bacillus coagulans is particularly useful as a composition for cleaning a surface. B. coagulans is non-pathogenic and is generally regarded as safe (i.e., GRAS classification) by the U.S. Federal Drug Administration (FDA) and the U.S. Department of Agriculture (USDA), and by those skilled in the art.

Bacillus coagulans is a non-pathogenic gram positive spore-forming bacteria that produces L(+) lactic acid (dextrorotatory) in fermentation conditions. It has been isolated from natural sources, such as heat-treated soil samples inoculated into nutrient medium (Bergey's Manual off Systemic Bacteriology, Vol. 2, Sneath, P.H.A., et al., eds., Williams & Wilkins, Baltimore, Md., 1986). Purified B. coagulans strains have served as a source of enzymes including endonucleases (e.g., U.S. Pat. No. 5,200,336); amylase (U.S. Pat. No. 4,980,180); lactase (U.S. Pat. No. 4,323,651); and cyclo-malto-dextrin glucano-transferase (U.S. Pat. No. 5,102,800). B. coagulans has been used to produce lactic acid (U.S. Pat. No. 5,079,164). A strain of B. coagulans (referred to as L. sporogenes; Sakaguti & Nakayama (ATCC 31284)) has been combined with other lactic acid producing bacteria and B. natto to produce a fermented food product from steamed soybeans (U.S. Pat. No. 4,110,477).

Bacterial species include Bacillus coagulans, e.g., Bacillus coagulans hammer, preferably Bacillus coagulans hammer strain Accession No. ATCC 31284, or one or more strains derived from Bacillus coagulans hammer strain Accession No. ATCC 31284 (e.g., ATCC Numbers: GBI-20, ATCC Designation Number PTA-6085; GBI-30, ATCC Designation Number PTA-6086; and GBI-40, ATCC Designation Number PTA-6087; see U.S. Pat. No. 6,849,256).

Bacillus coagulans was previously mis-characterized as a Lactobacillus and labeled as Lactobacillus sporogenes (See Nakamura et al. 1988. Int. J. Syst. Bacteriol. 38: 63-73). However, initial classification was incorrect because Bacillus coagulans produces spores and excretes L(+)-lactic acid through metabolism. Both of these characteristics provide key features to the utility of Bacillus coagulans. These developmental and metabolic aspects were the basis of reclassified as a Bacillus.

### Probiotic Activity of Bacillus Coagulans

Unsterilized surfaces may contain a variety of pathogenic microorganisms, such as bacterial, mycotic and yeast pathogens. It is well-documented clinically that many species of bacterial, mycotic and yeast pathogens possess the ability to cause a variety of gastrointestinal disorders including, but not limited to: disruption of normal gastrointestinal biochemical function, necrosis of gastrointestinal tissues, disruption of the bioabsorption of nutrients, and like conditions. The Bacillus coagulans supernatant described herein reduces the level of these pathogens on a surface. Thus, the compositions are useful in cleaning surfaces and preventing the conditions associated with infection by these aforementioned pathogens.

### Cleaning Compositions

The invention is directed to the surprising discovery that the supernatant of lactic acid-producing bacteria, particularly Bacillus species, reduces the amount of pathogenic microorganisms on a surface. In one aspect, the Bacillus coagulans supernatant contains secreted and/or extracellular compound(s) which possess anti-microbial activity. Alternatively, the Bacillus coagulans supernatant contains intracellular compound(s) which possess anti-microbial activity. These compound(s) are useful in the cleaning compositions according to the invention. Preferably, the Bacillus coagulans supernatant contains a protein which possess anti-microbial activity. Alternatively, the Bacillus coagulans supernatant contains an isolated small molecule which possess antimicrobial activity. Cell cultures are harvested as described below, and the culture supernatants are collected, by filtration or centrifugation, or both, and the resulting supernatant contains anti-microbial activity useful in the cleaning compositions of the present invention.

In one aspect, the Bacillus coagulans supernatant is undiluted prior to use in the cleaning compositions and methods of the invention. The invention also provides that the Bacillus coagulans supernatant is diluted prior to use in the cleaning compositions and methods of the invention. Exemplary dilutions of the Bacillus coagulans supernatant include about 1:2; about 1:5; about 1:10; about 1:50; about 1:100; about 1:500 or about 1:1,000. Preferably, the Bacillus coagulans supernatant is diluted in deionized water. Alternatively, the Bacillus coagulans supernatant is concentrated using standard methods prior to use in the compositions and methods of the invention. The Bacillus coagulans supernatant is concentrated about 2:1; about 5:1; about 10:1; about 25:1; about 50:1; about 75:1; about 100:1, about 500:1 or about 1,000:1.

In one aspect, the Bacillus coagulans supernatant is concentrated prior to being freeze-dried (lyophilized). Alternatively, the Bacillus coagulans supernatant is not concentrated prior to being freeze-dried. Optionally, the Bacillus coagulans supernatant is in the form of a dried powder and reconstituted (activated) prior to use in the cleaning compositions and methods of the invention. Preferably, the Bacillus coagulans supernatant is reconstituted in deionized water. More preferably, the supernatant is reconstituted in hot/warm water. In one aspect, the temperature of the hot or warm water for reconstitution of the supernatant is between 30° C. and 100° C.; between 32° C. and 90° C.; between 35° C. and 80° C.; between 40° C. and 70° C.; or between 50° C. and 60° C.

In another aspect, Bacillus coagulans supernatant in the form of a dried powder is applied directly to the surface to be cleaned prior to mixing with water. Optionally, the dried Bacillus coagulans supernatant is concentrated. Alternatively, the dried Bacillus coagulans supernatant is not concentrated. Preferably, water is added to the surface to be cleaned after the dried powder Bacillus coagulans supernatant is added to the surface.

In one aspect, the Bacillus coagulans supernatant is combined with one or more other surface-active disinfectants, surface-active agent (surfactant), or anti-microbial agents prior to use in the cleaning compositions and methods of the invention in order to enhance efficacy. Preferably, the surface-active disinfectants or antimicrobial agents are non-microbially-derived compounds. These non-microbially-derived, anti-microbial compounds include, but are not limited to: a quartenary ammonium chloride, an iodine or iodifer compound (e.g., Betadine™), a phenolic compound, an alcohol compound or tincture (e.g., ethanol, isopropyl, and the like). Suitable surface-active disinfectants include eythol alcohol, methyl alcohol, and quaternary ammonium chloride compounds. In one aspect, the Bacillus coagulans supernatant is present in a 1:1 ratio with the surface-active disinfectant described above. In another aspect, the Bacillus coagulans supernatant is combined 10:1; 5:1; or 2:1 with the surface-active disinfectant. Alternatively, the Bacillus coagulans supernatant is combined 1:2; 1:5; or 1:10 with the surface-active disinfectant.

In one aspect, the cleaning compositions described herein are sprayed onto the surface to reduce the amount of pathogenic microorganisms on the surface. Alternatively, the cleaning compositions are applied to a cloth or other dispersal device prior to contacting the surface. The invention provides for agitating or mopping the cleaning compositions, such as the Bacillus coagulans supernatant, on the surface to reduce the amount of pathogenic microorganisms on the surface. In another aspect, the Bacillus coagulans supernatant is applied to the surface as a dry powder.

This invention relates to cleaning, sanitizing, and/or sterilizing surfaces, e.g., medical devices and food service equipment. Exemplary surfaces include environmental surfaces, wood surfaces, metal surfaces, plastic surfaces, stainless steel surfaces and the like. Preferably, the surface is a surface associated with food-processing equipment. The cleaning compositions of the present invention are useful to sanitize hard surfaces (e.g., warewashing) and are particularly useful in the dairy and beverage industry for sanitizing substantially fixed, processing facilities such as pipelines and continuously operating homogenation or pasteurization apparatus, such as a milk line dairy, or a continuous brewing system. Alternatively, the surface is a surface associated with medical equipment or medical devices. The present invention relates to compositions and methods for cleaning and sterilizing medical equipment before or after use. Exemplary medical equipment or devices include operating or examination tables and tools, fiberscopes (medical-optic devices), such as gastrocameras and other types of endoscopes, including peritoneoscopes, thoracoscopes and arthoroscopes, and medical supplies, such as catheters and tubes, that have long ducts or hollow portions and that tend to be repetitively employed by being introduced into human bodies. Other suitable surfaces include surfaces in restaurants, homes, nursing homes, and private and public restrooms, e.g., desks, tables, chairs, stovetops, countertops, kitchen appliances, refrigerators, shelves, floors, toilets, sinks, bathtubs, windows, dog kennels, and baby equipment, such as baby changing tables, and diaper pails. Preferably, the cleaning compositions described by the invention are used as household cleaners.

The invention also provides for incorporation (embedding) of the cleaning compositions described herein into the surfaces of the invention. Preferably, the compositions are embedded into medical tools, such as those made of metal or synthetic or natural polymers.

In one aspect, the Bacillus coagulans supernatant remains on the surface as a liquid film and is allowed to dry for a period of time. Alternatively, the Bacillus coagulans supernatant remains on the surface as a liquid film for a period of time before removing the supernatant from the surface. Optionally, the period of time is between about 15 seconds and about 30 minutes, e.g., between about 30 seconds and about 15 minutes; between about 1 minute and about 10 minutes; or between about 2 minutes and about 8 minutes. Preferably, the period of time is about 5 minutes.

### EXAMPLE 1

### Preparation of Bacillus Coagulans Cultures

Bacillus coagulans Hammer bacteria (ATCC Accession No. 31284) was inoculated and grown to a cell density of about 10⁸ to 10⁹ cells/ml in nutrient broth containing 5 g Peptone, 3 g Meat extract, 10-30 mg MnSO₄, and 1,000 ml distilled water, adjusted to pH 7.0, using a standard airlift fermentation vessel at 30° C. The range of MnSO₄ acceptable for sporulation is 1 mg/l to 1 g/l. The vegetative cells can actively reproduce up to 45° C., and the spores are stable up to 90° C. After fermentation, the B. coagulans bacterial cells or spores are collected using standard methods (e.g., filtration, centrifugation). As described below, the supernatant from the cell culture is collected and used in the cleaning compositions and methods of the invention.

### EXAMPLE 2

### Preparation of B. Coagulans and P. Lindbergii Supernatants

One liter cultures of either Bacillus coagulans or Pseudomonas lindbergii were prepared as described above. The culture was first autoclaved for 30 minutes at 250° F., and then centrifuged at 10,000 revolutions per minute (r.p.m.) for 15 mm. The resulting supernatant was collected and through a 0.45 μm filter. The filtrate was collected and further filtered through a 0.2 μm Nalgene vacuum filter. The resulting final filtrate was then collected (an approximate volume of 900 ml) to form a liquid containing an extracellular product which was to be quantitatively analyzed and utilized in the subsequent inhibition studies.

### EXAMPLE 3

### Bacillus Coagulans Supernatant Kills Pathogenic Microorganisms

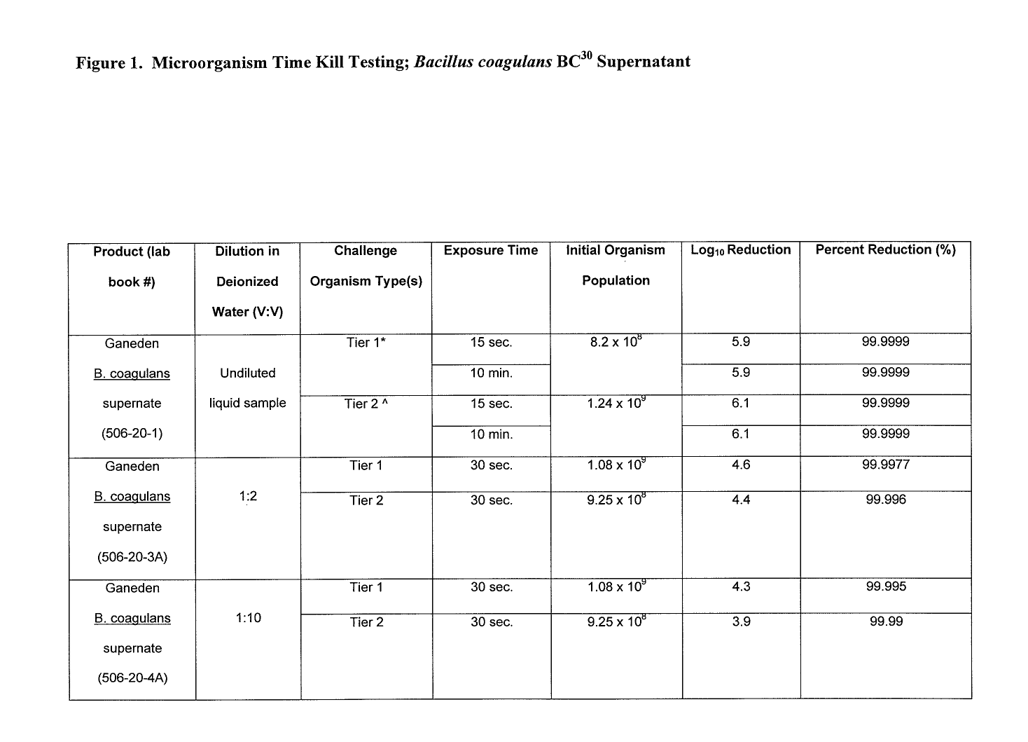

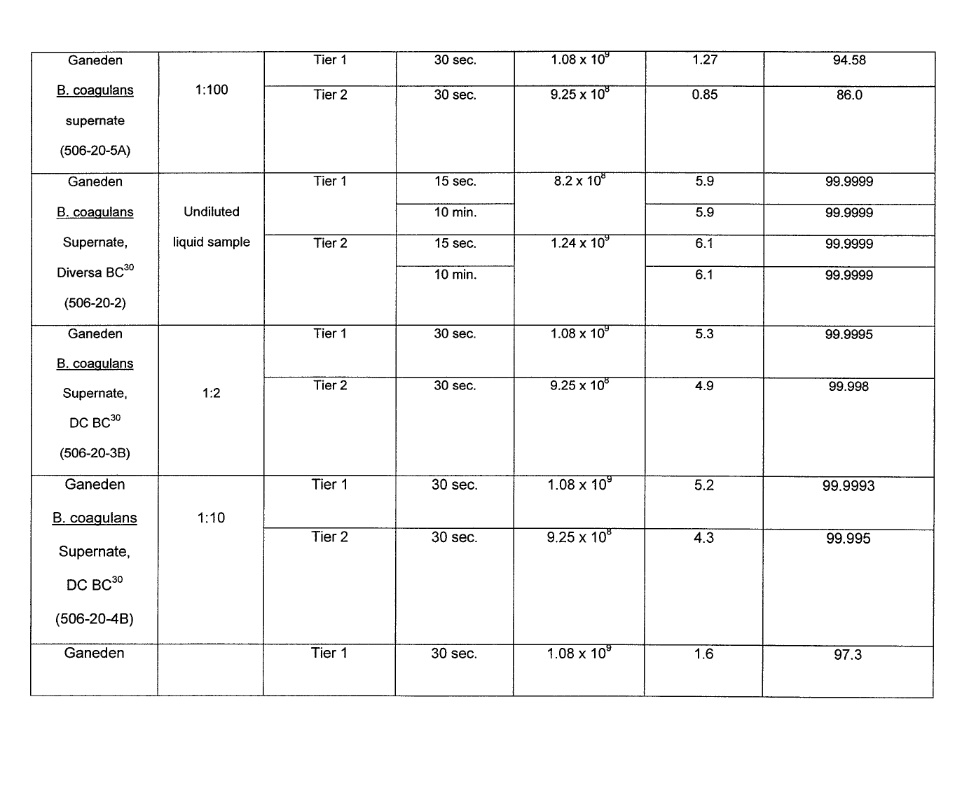

This study was performed in order to determine the ability of Bacillus coagulans supernatant to kill pathogenic microorganisms. Supernatant was harvested from Bacillus coagulans (506-20-1) and BC³⁰ (Bacillus coagulans-30; GBI-30). Bacillus coagulans (506-20-1) produces slightly more lactic acid compared to BC³⁰. Each supernatant was diluted in deionized water as indicated in FIG. 1. The Bacillus coagulans supernatant was then added to a surface comprising either Tier 1 or Tier 2 pathogenic microorganisms. Tier 1 pathogenic microorganisms included the following: E. coli (ATCC#11229), S. aureus (ATCC#6538), E. faecium (VRE), MDR (ATCC#51559), and S. marcescens (ATCC#14756). Tier 2 pathogenic microorganisms included the following: S. aureus MRSA (ATCC#33591), Proteus mirabilis (ATCC#7002), K. pneumoniae (ATCC#13883), and S. epidermidis (ATCC# 12228). The pathogenic microorganisms were exposed to the Bacillus coagulans supernatant for a period of time ranging from 15 seconds to 10 minutes. As shown in FIG. 1, undiluted liquid sample of BC³⁰ supernatant killed 99.9999% of both Tier 1 and Tier 2 pathogenic microorganisms after an exposure time of 15 seconds. Similarly, a 1:100 diluted sample of BC³⁰ supernatant killed about 97% of both Tier 1 and Tier 2 pathogenic microorganisms after an exposure time of 30 seconds. These data demonstrate that Bacillus coagulans supernatant kills pathogenic microorganisms.

### EXAMPLE 4

### Bacillus Coagulans Supernatant has Antimycotic Activity

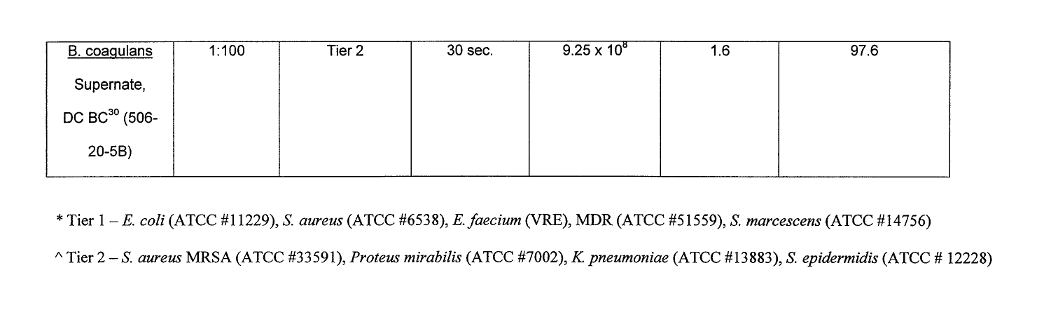

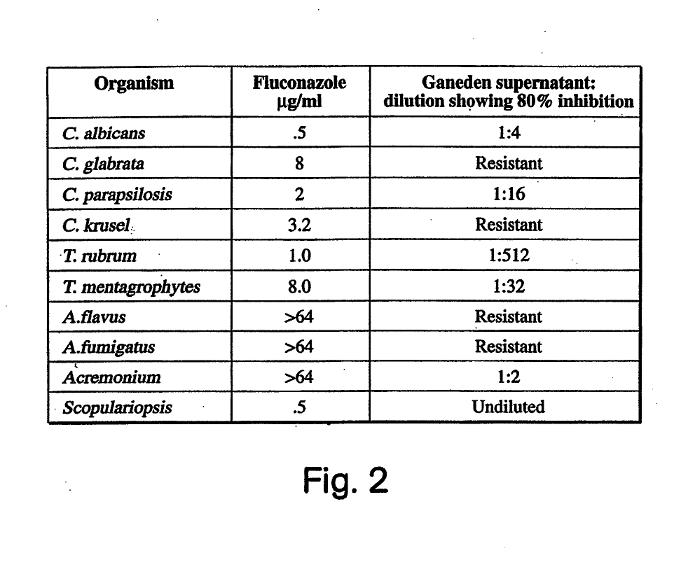

In an additional assay, a comparison of the anti-mycotic, Fluconazole with Bacillus coagulans supernatant in the inhibition of various bacterial, fungal, and yeast species, was performed. As illustrated in FIG. 2, these supernatants were effective in inhibiting a majority of the organisms against which they were tested. Serial dilutions of the Bacillus coagulans supernatant were performed with RPMI medium and inhibition was determined at 80% in accordance with the NCCLS standard for anti-fungal susceptibility. Specifically, the results demonstrated that Trichophyton rubrum was totally inhibited by undiluted supernatant, and 1:2, 1:4, 1:8, 1:16, 1:32, 1:64, 1:128, and 1:256 serial dilutions, and the organism was 80% inhibited by the compound diluted 1:512 with RPMI medium.

Trichophyton mentagrophytes was totally inhibited by the undiluted supernatant, and 1:2, 1:4, 1:8, and 1:16 serial dilutions, and the organism was 80% inhibited by the supernatant diluted 1:32 with RPMI medium. Candida parapsilosis was totally inhibited by the undiluted supernatant and 1:2, 1:4, 1:8, 1:16, 1:32, 1:64, 1:128, and 1:256 serial dilutions, and the organism was 80% inhibited by the supernatant diluted 1:16 with RPMI medium. Candida albicans was totally inhibited by the undiluted supernatant and a 1:2 dilution, and the organism was 80% inhibited by the supernatant diluted 1:4 with RPMI medium. Acremonium sp. was totally inhibited by the undiluted supernatant and was 80% inhibited by the supernatant diluted 1:2 with RPMI medium. Scopulariopis sp. was 80% inhibited by the undiluted supernatant, but was uninhibited by any of the serial dilutions of the supernatant. The supernatant showed no inhibitory activity against Candida glabrata, Candida krusel, or the two Aspergillus species. Thus, the supernatant was demonstrated to possess marked inhibitory activity, in a wide variety of dilutions, against a majority of the tested organisms. Moreover, the Bacillus coagulans supernatant appeared to be extremely effective against dermatophytes (e.g., Trichophyton sp.), which are a causative organism in many mammalian dermal diseases.

What is claimed is:

1. A method for cleaning a surface comprising contacting said surface with a Bacillus coagulans supernatant composition, wherein said Bacillus coagulans supernatant reduces the concentration of pathogenic or opportunistic microorganisms on said surface.

2. The method of claim 1, wherein said surface is an environmental surface.

3. The method of claim 1, wherein said Bacillus coagulans supernatant remains on said surface as a liquid film and is allowed to dry for a period of time.

4. The method of claim 3, wherein said period of time is between 15 seconds and 10 minutes.

5. The method of claim 3, wherein said period of time is 5 minutes.

6. The method of claim 1, further comprising agitating said Bacillus coagulans supernatant after contact with said surface.

7. The method of claim 1, wherein said pathogenic microorganisms are selected from the group consisting of Escherichia, Enterococcus, Staphylococcus, Clostridium, Campylobacter, Pseudomonas, and Streptococcus.

8. The method of claim 1, wherein said pathogenic microorganisms are selected from the group consisting of Escherichia coli, Staphylococcus aureus, Vancomycin resistant Enterococcus, Bacillus cereus, multi-drug resistant Enterococcus, Enterococcus faecium, Serratia marcescens, Proteus mirabilis, Klebsiella pneumoniae, and Staphylococcus epidermidis.

9. The method of claim 1, wherein said pathogenic microorganisms are fungal pathogens selected from the group consisting of Candida and Trichophyton.

10. The method of claim 1, wherein said Bacillus coagulans supernatant is undiluted.

11. The method of claim 1, wherein said Bacillus coagulans supernatant is diluted 1:2.

12. The method of claim 1, wherein said Bacillus coagulans supernatant is diluted 1:10.

13. The method of claim 1, wherein said Bacillus coagulans supernatant is diluted 1:100.

14. The method of claim 1, wherein said Bacillus coagulans supernatant is concentrated at a ratio selected from the group consisting of 100:1, 75:1, 50:1, 25:1, 10:1, 5:1, and 2:1.

15. The method of claim 1, wherein said supernatant is dried into a powder and reconstituted in hot or warm water before contacting said surface.

16. The method of claim 1, wherein said supernatant is combined with one or more other surface-active disinfectants prior to contacting said surface.

17. The method of claim 16, wherein said surface-active disinfectants are selected from the group consisting of ethyl alcohol, methyl alcohol, and quaternary ammonium chloride compounds.

18. The method of claim 1, wherein said Bacillus coagulans is Bacillus coagulans hammer strain Accession No. ATCC 31284.

19. The method of claim 1, wherein said Bacillus coagulans is GBI-30 strain (ATCC Designation Number PTA-6086).

20. The method of claim 1, wherein said Bacillus coagulans is GBI-20 strain (ATCC Designation Number PTA-6085).

21. The method of claim 1, wherein said Bacillus coagulans is GBI-40 strain (ATCC Designation Number PTA-6087).

22. The method of claim 1, wherein said cleaning disinfects said surface.

23. The method of claim 1, wherein said cleaning sterilizes said surface.

24. The method of claim 1, wherein said Bacillus coagulans supernatant reduces the level of pathogenic microorganisms on said surface by at least 90%.

25. A composition for cleaning a surface comprising an antimicrobial amount of a secreted product of Bacillus coagulans.

26. A composition for cleaning a surface comprising an antimicrobial amount of a supernatant of Bacillus coagulans.

27. A composition for cleaning a surface comprising an antimicrobial amount of a lysate of Bacillus coagulans.

28. A composition for cleaning a surface comprising an antimicrobial amount of a peptide or organic molecule isolated from Bacillus coagulans supernatant.

29. A composition for cleaning a surface comprising an antimicrobial amount of a supernatant of Bacillus coagulans and a surface-active agent.

30. The composition of claim 29, wherein said surface-active agent is selected from the group consisting of ethyl alcohol, methyl alcohol, and a quaternary ammonium chloride compound.

31. The composition of claim 29, wherein said composition is in the form of dry powder or pellets.

32. The composition of claim 29, wherein said composition is in the form of a liquid or suspension.
